(19) United States
(12) Patent Application Publication
(10) Pub. No.: US 2023/0162812 A1
(43) Pub. Date: May 25, 2023

(54) CANCER DETECTION USING MITOCHONDRIAL GENOME

(71) Applicant: PetDx, Inc., La Jolla, CA (US)

(72) Inventors: Kristina Kruglyak, La Jolla, CA (US); Carlos A. Ruiz Perez, La Jolla, CA (US)

(21) Appl. No.: 18/056,653

(22) Filed: Nov. 17, 2022

Related U.S. Application Data
(60) Provisional application No. 63/264,433, filed on Nov. 22, 2021.

(51) Int. Cl. G16B 20/10; C12Q 1/6869; G16B 45/00

(52) U.S. Cl. CPC G16B 20/10; C12Q 1/6869; G16B 45/00

(57) ABSTRACT

The methods, systems, and compositions provided herein allow improved methods for identifying cancer by measuring normalized truncated average sequencing depth from a mitochondrial chromosome in a population of samples in order to improve identification of cancer samples.

### CROSS REFERENCE TO RELATED APPLICATIONS

This application claims the benefit of priority to U.S. Provisional Application No. 63/264,433, filed Nov. 22, 2021. The disclosure of the above-referenced application is hereby incorporated by reference in its entirety.

### FIELD

The present disclosure relates to methods for identifying cancer in a sample by measuring a normalized truncated average sequencing depth (NTAD) of a mitochondrial chromosome (ChrM) in cancer subjects, wherein the measured NTAD of ChrM above or below a threshold is indicative of a cancer sample.

### BACKGROUND

Cancer remains a predominant health care concern, with an estimated incidence of 442.4 per 100,000 individuals, and an estimated death rate of 158.3 per 100,000 individuals per year. Among the most common cancers are breast cancer, lung and bronchus cancer, prostate cancer, colon and rectum cancer, melanoma of the skin, bladder cancer, non-Hodgkin lymphoma, kidney and renal pelvis cancer, endometrial cancer, leukemia, pancreatic cancer, thyroid cancer, and liver cancer. Prostate, lung, and colorectal cancers account for an estimated 43% of all cancers diagnosed in men. For women, 50% of all diagnosed cancers are breast, lung, and colorectal.

Current methods of cancer diagnosis include imaging, radiolabeling, and biopsies. A liquid biopsy is a type of diagnostic methodology test done on a blood sample to detect, among other things, cell free DNA (cfDNA) that is circulating in the blood. (Chibuk, Front Vet Sci, 8, 2021). Somatic alterations that are present in the cfDNA can be detected and used to screen for the presence of tumor cells in the body. Liquid biopsy has also been used globally in noninvasive prenatal testing for the screening of fetal chromosomal aneuploidies and has led to a considerable reduction in invasive prenatal testing, such as use of amniocentesis. Liquid biopsies for organ transplant patients have been used to monitor graft dysfunction. Cancer liquid biopsies have been used for the selection of targeted therapies and monitoring of disease progression. There is a need to further improve the performance, such as sensitivity, specificity, recall, and/or precision of liquid biopsy to extend applicability of the techniques to other cancer types and other species.

### SUMMARY

Described herein are methods and compositions for the detection, diagnosis, and screening of cancer in subjects. In some embodiments, the methods disclosed herein are capable of identifying a cancer sample where other methods known in the art were incapable of identifying cancer.

Some embodiments provided herein relate to methods of detecting cancer in a subject. In some embodiments, the methods include collecting a liquid biopsy sample from the subject. In some embodiments, the methods include determining ChrM sequencing depth. In some embodiments, the methods include an exclusion of hypervariable regions from ChrM. In some embodiments, the methods include truncating the ChrM sequencing depth. In some embodiments, the methods include calculating a relative quantity of ChrM DNA by comparison to total cfDNA. In some embodiments, the methods include applying a threshold. In some embodiments, applying the threshold isolates a subject with cancer from a cancer free subject.

In some embodiments, the relative quantity of ChrM DNA is a normalized truncated average sequencing depth (NTAD). In some embodiments, the ChrM sequencing depth is an average sequencing depth. In some embodiments, the relative quantity of ChrM DNA is a ChrM rate. In some embodiments, the methods further include measuring log10 of NTAD. In some embodiments, the NTAD is scaled by a factor prior to log10 transformation. In some embodiments, the scale factor is 10, 100, 1,000, 10,000, 100,000, or 1,000,000. In some embodiments, the ChrM sequencing depth is normalized by measuring ChrM reads per base per total reads. In some embodiments, the ChrM average sequencing depth is normalized by measuring ChrM sequencing depth per base per total reads.

In some embodiments, truncating the ChrM sequencing depth comprises removing outliers from a distribution of ChrM per-base sequencing depth. In some embodiments, the outliers comprise a top 10% and a bottom 10% of measured ChrM per-base sequencing depth. In some embodiments, the threshold is 1, 2, or 3 standard deviations from the mean value of NTAD of healthy subjects. In some embodiments, the threshold is more than 3 standard deviations from the mean value of NTAD of healthy subjects. In some embodiments, the threshold is based on a modeled cumulative distribution function (CDF) quantile. In some embodiments, the modeled CDF quantile is 0.01, 0.005, 0.001, or 0.0001.

In some embodiments, the methods further include applying a statistical analysis to determine whether the relative quantity of ChrM DNA is distributed normally. In some embodiments, the statistical analysis is a Q-Q test or a Shapiro-Wilk test. In some embodiments, the methods further include determining precision/recall to determine a performance of the method and/or a relation between true positives, false positives, true negatives, and false negatives.

In some embodiments, the sample comprises circulating cell free DNA (cfDNA) or fragments thereof In some embodiments, the cancer sample is leukemia, lymphoma, testicular tumor, spinal meningioma, multilobular osteochondrosarcoma, soft tissue sarcoma, squamous cell carcinoma, mammary cancer, mast cell tumors, bladder cancer, osteosarcoma, or hemangiosarcoma. In some embodiments, the subject is a mammal. In some embodiments, the subject is canine, feline, equine, or human.

### BRIEF DESCRIPTION OF THE DRAWINGS

FIG. 1 depicts a circular representation of the Canis lupus familaris mitochondrial chromosome. The outer circle shows protein-coding, tRNA and rRNA genes with their respective annotations. The middle circle shows the sequencing depth along the chromosome and shows a decrease in sequencing depth in the hypervariable region excluded from the ChrM NTAD calculation. Finally, the inner circle shows the GC content per window along the mitochondrial chromosome.

FIG. 2A depicts a scatter plot (top) and a bar graph (bottom), which show a distribution of ChrM rates as the total number of reads mapping to the ChrM normalized by a total number of reads in the sequencing library. In the scatter plot, healthy subjects are represented by a cancer status of 0, and cancer subjects are represented by a cancer status of 1.

FIG. 2B depicts log10 transformations of ChrM rates as the total number of reads mapping to the ChrM, normalized by the total number of reads in the sequencing library, and converted to a log10 scatter plot (top) and a log10 bar graph (bottom). In the scatter plot, healthy subjects are represented by a cancer status of 0, and cancer subjects are represented by a cancer status of 1.

FIG. 3 is a statistical Q-Q probability plot of log10 ChrM rates along with the p-value of the associated Shapiro-Wilk normality test.

FIG. 4A depicts a non-normalized line graph of sequencing depth per base along the mitochondrial chromosome position for three subjects. Subject 101 and 201 have identical ChrM rates of 0.006, and subject 301 has a ChrM rate of 0.003.

FIG. 4B depicts a line graph of sequencing depth per base for three subjects normalized by sequencing effort. Subject 101 and 201 have identical ChrM rates of 0.006, and subject 301 has a ChrM rate of 0.003.

FIG. 5 shows a series of line graphs that depict the sorted per-base sequencing depth of a healthy subject (left), and two cancer-positive subjects (middle and right plots).

FIG. 6 shows a series of line graphs that depict the sorted per-base sequencing depth of a healthy subject (left), and two cancer-positive subjects (middle and right plots). The shaded areas represent the lower and upper 10% of bases that are removed (truncated) from the distribution.

FIG. 7 is a dot plot of ChrM normalized truncated average sequence depths (NTAD) of healthy subjects (cancer status NEGATIVE) compared to cancer subjects (cancer status POSITIVE).

FIG. 8 is a histogram showing the distribution of normalized truncated average sequence depth (NTAD) of healthy subjects.

FIG. 9 is a statistical Q-Q probability plot of normalized truncated average sequence depth (NTAD) of healthy subjects along with the p-value of the associated Shapiro-Wilk normality test.

FIG. 10 is a scatter plot showing the relation between the normalized truncated average sequence depth (NTAD) of healthy and cancer subjects (x-axis) and their associated fragment insert size standard deviation. The plot indicates that higher fragment insert size standard deviations are associated with higher NTAD values.

FIG. 11 depicts a histogram of normalized truncated averaged sequence depth (NTAD) values of healthy subjects with fragment insert size standard deviations (IS SD) below 400. IN addition, the dashed line indicates a normal model fitted to all healthy subjects (including those with IS SD above 400), while the solid line indicates a normal model fitted to healthy subjects with IS SD below 400.

FIG. 12 depicts a dot plot of healthy and cancer subjects (cancer status 0 or 1, respectively; primary y-axis) and the fitted normal distribution along with its mean and mean±one, two, and three standard deviations (secondary y-axis).

FIG. 13 depicts a precision/recall curve indicating that a lower threshold of three standard deviations below the mean results in a precision of one (no false positives), while allowing identification of cancer positive samples.

FIG. 14 depicts a dot plot of healthy and cancer subjects (cancer status 0 or 1, respectively) along with the mean and standard deviations around the mean derived from the fitted normal distribution. Some examples of predicted cancer positive subjects and their cancer types are highlighted.

FIG. 15 depicts a precision/recall curve using different quantile values and their associated NTAD values as thresholds. Note that NTAD values below −1.98244, corresponding to values below the mean −2 standard deviations result in a precision of one (no false positives), while allowing identification of cancer positive samples.

FIG. 16 is a dot plot of healthy and cancer subjects (cancer status 0 or 1, respectively) in a training dataset along with the selected NTAD threshold for cancer prediction (−2.36497, corresponding to 0.01% quantile in the distribution). Some examples of cancer-positive subjects and their diagnosed cancer are highlighted.

FIG. 17 is a dot plot of healthy and cancer subjects (cancer status 0 or 1, respectively) in a testing dataset along with the selected NTAD threshold for cancer prediction (−2.36497, corresponding to 0.01% quantile in the distribution). Some examples of cancer-positive subjects and their diagnosed cancer are highlighted.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein. All references cited herein are expressly incorporated by reference herein in their entirety and for the specific disclosure referenced herein.

Provided herein are methods, systems, and compositions for identifying cancer samples by calculating normalized average sequencing depth (NTAD) of mitochondrial chromosome (ChrM). In some embodiments, the methods include measuring ChrM sequencing reads. In some embodiments, the methods include measuring ChrM rate. In some embodiments, the methods include applying a threshold to the ChrM rate or NTAD, thereby identifying samples that are cancer samples from among a population of samples, some of which are healthy samples.

Methods and compositions provided herein improve the detection, diagnosis, staging, screening, treatment, and management of cancer in subjects, particularly in humans, mammals, and other types of subjects.

It should be realized that the analysis described herein may be part of a larger diagnostic suite used to determine a subject's overall health. For example, the analysis of ChrM sequencing depth in a subject may be used simultaneously or sequentially with other methods for detection, diagnosis, staging, screening, treatment, and management of cancer including additional genetic variance analysis. These procedures may be useful to detect a variety of cancers, including, but not limited to, leukemia, lymphoma, testicular tumor, spinal meningioma, multilobular osteochondrosarcoma, soft tissue sarcoma, squamous cell carcinoma, mammary cancer, mast cell tumors, bladder cancer, osteosarcoma, hemangiosarcoma or a variety of other cancers afflicting subjects.

Some embodiments provided herein relate to methods for identifying a cancer sample. In some embodiments, the methods include measuring the rate of the ChrM as the total number of reads mapping to ChrM, normalized by the total number of reads in the dataset. In some embodiments, certain hypervariable regions from ChrM can be excluded to avoid extremely low sequencing depth values. In some embodiments, the log10 of the ChrM rates are estimated in order to centralize skewness of the distribution.

In some embodiments, the methods include obtaining or having obtained a biological sample from a subject that has or is suspected of having cancer. In some embodiments, the sample is a liquid biopsy sample, such as a blood sample. In some embodiments, the sample includes cfDNA. In some embodiments, the sample is provided in an amount of less than 10 mL, such as 10 mL, 9 mL, 8 mL, 7 mL, 6, mL, 5 mL, 4 mL, 3 mL 2 mL, 1 mL, 500 μL, 250 μL, 100 μL or an amount within a range defined by any two of the aforementioned values. In some embodiments, the sample includes DNA in an amount of less than or equal to 10 μg, such as 10 μg, 5 μg, 1 μg, 500 ng, 100 ng, 50 ng, 10 ng, 5 ng, 1 ng, 500 pg, 100 pg, 50 pg, 10 pg, 9, pg, 8 pg, 7 pg, 6 pg, 5 pg, 4 pg, 3 pg, 2 pg, or 1 pg, or in an amount within a range defined by any two of the aforementioned values. In some embodiments, the method includes purifying the DNA from the sample. Purifying the DNA may be accomplished using DNA purification techniques, including, for example extraction techniques, precipitations, chromatography, bead-based methods, or commercially available kits for DNA purification.

As used herein, the term “cfDNA” has its ordinary meaning as understood light of the specification, and refers to circulating cell free DNA, which includes DNA fragments released to the blood plasma. cfDNA can include circulating tumor deoxyribonucleic acid (ctDNA).

In some embodiments, the methods include measuring the NTAD as the truncated average sequencing depth of ChrM, normalized by the total number of reads in the dataset. In some embodiments, the log10 of the NTAD are estimated in order to centralize skewness of the distribution. In some embodiments, the NTAD is scaled by a factor prior to performing a log10 transformation. In some embodiments, the scale factor may be, for example, a scale of 10, 100, 1,000, 10,000, 100,000, or 1,000,000. In some embodiments, low NTAD values are observed in datasets of a population of samples, wherein the population of samples includes both healthy and cancer samples.

In some embodiments, a threshold is established from the log10 data to identify cancer samples. In some embodiments, the log10 ChrM rates or the log10 NTAD values are tested for normality in order to effectively establish thresholds using means and standard deviations. In some embodiments, the normal distribution of log10 ChrM rates or of log10 NTAD is tested using a statistical analysis. In some embodiments, the statistical analysis is a Q-Q plot and/or a Shapiro-Wilk test. In some embodiments, a normal distribution is fitted to the log10 ChrM rates or the log10 NTAD in order to effectively establish thresholds using distribution quantiles.

As used herein, a Q-Q test, or a quantile vs quantile plot, has its ordinary meaning as understood in light of the specification and refers to a statistical analysis for determining deviation of a normal distribution by plotting theoretical quantiles against actual quantiles of a variable. A straight line on a Q-Q plot is indicative of normal distribution.

As used herein, a Shapiro-Wilk test has its ordinary meaning as understood in light of the specification and refers to a statistical analysis for determining normal distribution by assuming that the population is normally distributed, and a p value is less than a certain threshold is indicative that the data tested are not normally distributed.

In some embodiments, the methods further include sorting the per-base sequencing depth to determine whether there is an increase from bases with no coverage to highly covered bases. In some embodiments, extreme values (or outliers) are removed and average sequencing depth is calculated.

Thus, in some embodiments, the methods further include removing a designated number of top and/or bottom values of the distributions. In some embodiments, the top and/or bottom values of the distributions are removed, such as the top and/or bottom 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 6%, 7%, 8%, 9%, 10%, 11%, 12%, 13%, 14%, 15%, 20%, or 25%, or an amount within a range defined by any two of the aforementioned values. In some embodiments, the methods include removing the top 10% and bottom 10% of distributions. In some embodiments, the values of distributions to be removed is determined by making a determination of which percentage is an extreme value (or an outlier value), using any type of statistical analysis. In some embodiments, removal of the top and/or bottom values of distributions is referred to herein as truncated average sequencing depth (TAD).

Normality tests in the form of Q-Q plots and Shapiro-Wilk normality tests may be used to determine whether the data is normally distributed. In some embodiments, where the data is not normally distributed, normal distributions are achieved by setting thresholds based on standard deviations to perform transformations in the data to approximate to a normal distribution, such as a normalized truncated average sequencing depth (NTAD). In some embodiments logs, square-root, cubic-root, or z-score normalizations, or combinations thereof are used for normalizations.

In some embodiments, thresholds are applied to the NTAD. In some embodiments, the threshold is set at 1, 2, or 3 standard deviations from the mean. In some embodiments, the threshold is set at greater than 3 standard deviations from the mean. In some embodiments, the threshold is based on a quantile of a modeled cumulative distribution function (CDF). In some embodiments, the modeled CDF quantile is 0.01, 0.005, 0.001, or 0.0001. In some embodiments, the threshold is determined where there are no false positives, and/or where cancer positive samples are identified.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

As used herein, “a” or “an” can mean one or more than one.

As used herein, the term “about” or “approximately” has its usual meaning as understood by those skilled in the art and thus indicates that a value includes the inherent variation of error for the method being employed to determine a value, or the variation that exists among multiple determinations.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm”.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “function” and “functional” as used herein have their plain and ordinary meaning as understood in light of the specification, and refer to a biological, enzymatic, or therapeutic function.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 81, 82, 83, 84, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, or living cells which make up these living organisms, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

As used herein, “nucleic acid”, “nucleic acid molecule”, or “nucleotide” refers to polynucleotides or oligonucleotides such as deoxyribonucleic acid (DNA) or ribonucleic acid (RNA), including chromosomal DNA, oligonucleotides, fragments generated by the polymerase chain reaction (PCR), and fragments generated by any of ligation, scission, endonuclease action, exonuclease action, and by synthetic generation. Nucleic acid molecules can be composed of monomers that are naturally occurring nucleotides (such as DNA and RNA), or analogs of naturally occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally occurring or modified nucleic acid bases attached to a polyamide backbone. Nucleic acids can be either single stranded or double stranded.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “gene” as used herein has its plain and ordinary meaning as understood in light of the specification, and generally refers to a portion of a nucleic acid that encodes a protein or functional RNA; however, the term may optionally encompass regulatory sequences. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs and miRNAs. In some cases, the gene includes regulatory sequences involved in transcription, or message production or composition. In other embodiments, the gene comprises transcribed sequences that encode for a protein, polypeptide, or peptide. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide, or peptide encoding sequences, etc. In this respect, the term “gene” is used for simplicity to refer to a nucleic acid comprising a nucleotide sequence that is transcribed, and the complement thereof. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express or may be adapted to express using nucleic acid manipulation technology, proteins, polypeptides, domains, peptides, fusion proteins, mutants and/or such like.

The terms “cancer” and “cancerous” have their ordinary meaning as understood in light of the specification and refer to or describe the physiological condition in animals that is typically characterized by unregulated cell growth. A “tumor” comprises one or more cancerous cells. In some embodiments, the tumor is a solid tumor. There are several main types of cancer. Carcinoma is a cancer that originates from epithelial cells, for example skin cells or lining of intestinal tract. Sarcoma is a cancer that originates from mesenchymal cells, for example bone, cartilage, fat, muscle, blood vessels, or other connective or supportive tissue. Leukemia is a cancer that originates in hematopoietic cells, such as the bone marrow, and causes large numbers of abnormal blood cells to be produced and enter the blood. Lymphoma and multiple myeloma are cancers that originate in the lymphoid cells of lymph nodes. Central nervous system cancers are cancers that originate in the central nervous system and spinal cord.

The terms “individual”, “subject”, “host,” or “patient” as used herein have their usual meaning as understood by those skilled in the art and thus includes a human or a non-human mammal. The term “mammal” is used in its usual biological sense. Thus, it specifically includes, but is not limited to, primates, including simians (chimpanzees, apes, monkeys), humans, cattle, horses, sheep, goats, swine, rabbits, dogs, cats, rodents, rats, mice, or guinea pigs.

As used herein, the term mitochondrial chromosome, or ChrM, has its ordinary meaning as understood in light of the specification and refers to a sequence of mitochondrial DNA, which is DNA present in or originating in the mitochondria.

As used herein, the term mitochondrial chromosome rate, or ChrM rate, has its ordinary meaning as understood in light of the specification and refers to a number of ChrM reads divided by the number of total reads. In some embodiments, the methods, systems, and compositions provided herein relate to analyzing the rates of ChrM in cancer and non-cancer samples to determine whether a lower and/or upper threshold may be obtained to allow differentiation and identification of a cancer sample.

As used herein, the term sequencing depth has its ordinary meaning as understood in light of the specification and refers to the number of times a base in a chromosome position is covered by a read, and can be measured as reads per base, or reads per base per total bases along the chromosome position. In some embodiments, the sequencing depth is obtained using next generation sequencing (NGS).

As used herein, “next generation sequencing” or “NGS” refers to a procedure similar to capillary electrophoresis-based sequencing in which DNA polymerase catalyzes the incorporation of fluorescently labeled deoxyribonucleotide triphostphates (dNTPs) into a DNA template strand during sequential cycles of DNA synthesis. During each cycle, at the point of incorporation, the nucleotides are identified by fluorophore excitation. Instead of sequencing a single DNA fragment, the process extends across millions of fragments in a massively parallel manner.

In some embodiments, the NGS workflow may include the steps of: (1) preparing a sequencing library by random fragmentation of DNA or cfDNA in the sample, followed by 5′ and 3′ adapter ligation. Alternatively, “tagmentation” may be used, which combines the fragmentation and ligation reactions into a single step to increase the efficiency of the library preparation step. Adapter-ligated fragments are then PCR amplified and gel purified; (2) loading the library into a flow cell for cluster generation, where fragments are captured by surface-bound oligos complementary to the library adapters. Each fragment may be amplified into distinct, clonal clusters through bridge amplification. When cluster generation is completed, the templates are ready for sequencing; (3) incorporating a first base by adding sequencing reagents, including fluorescently labeled nucleotides. The flow cell may be imaged and the emission from each cluster may be recorded. The emission wavelengths and intensities are used to identify the bases; (4) aligning newly identified sequence reads to a reference genome. After alignment, differences between the reference genome and the newly sequenced reads can be identified.

As used herein the term normalized truncated average sequencing depth, or NTAD, has its ordinary meaning as understood in light of the specification, and refers to truncated average sequencing depth (TAD) of ChrM divided by total reads, where TAD refers to average sequencing depth of ChrM to which a truncation has been applied to remove a designated number of top and/or bottom values of the measured sequencing depths.

As used herein, the term threshold has its ordinary meaning as understood in light of the specification, and refers to a set limit for making a determination or identification of whether a particular sample is a cancer sample. In some embodiments, a threshold is set to increase precision in identification of a cancer sample, such that no false positives are identified, while concomitantly allowing identification of cancer positive samples. In some embodiments, the threshold is a threshold of two or three standard deviations from the mean. In some embodiments, the threshold is a threshold of greater than three standard deviations from the mean.

### EXAMPLES

Embodiments of the present invention are further defined in the following Examples. It should be understood that these Examples are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims. The disclosure of each reference set forth herein is incorporated herein by reference in its entirety, and for the disclosure referenced herein.

### Example 1

### Measuring Mitochondrial Chromosome Rate

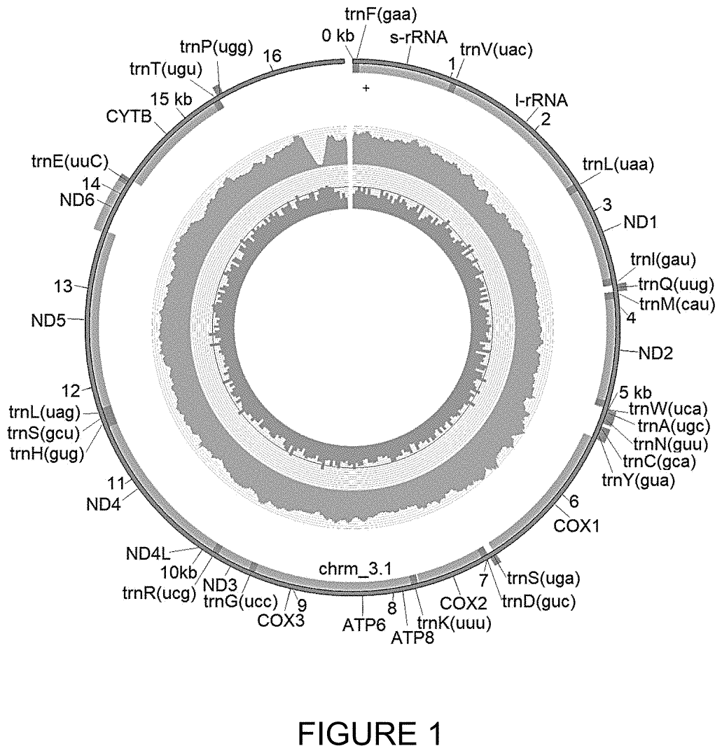

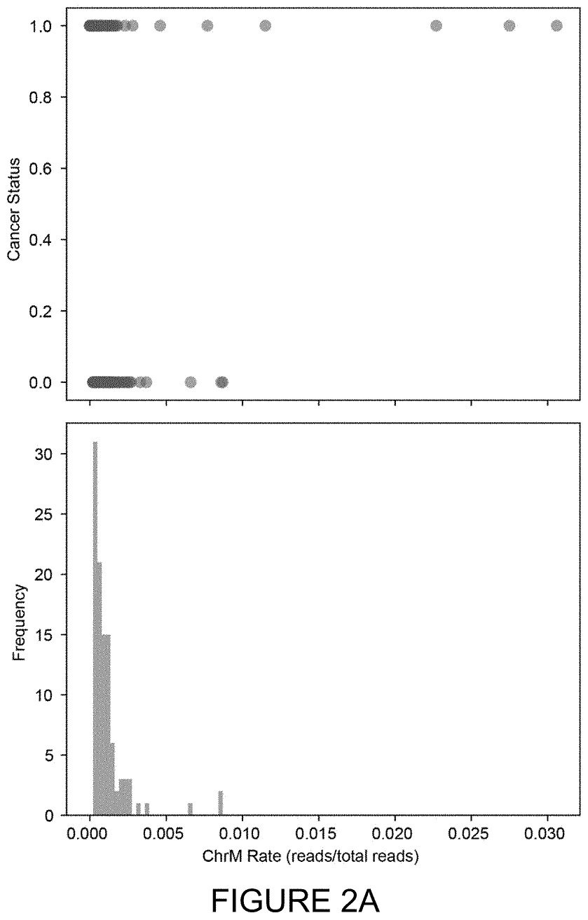

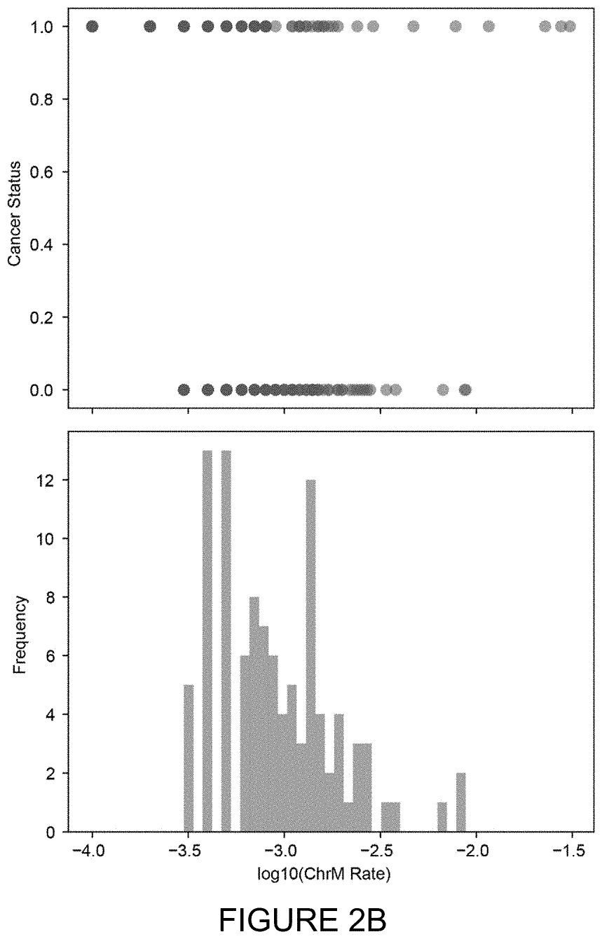

A training data set including a population of samples, including both healthy and cancer samples, was provided. The distribution of mitochondrial chromosome (ChrM) rate was determined by measuring the ChrM rate in cancer samples compared to non-cancer samples. A hypervariable region occurring in the mitochondrial chromosome results in low numbers of reads mapping to this regions (FIG. 1). Therefore, this region is excluded from any calculation. The distribution of rates in training datasets was measured, as shown in FIG. 2A, with the distribution skewed to the right, with some samples showing larger ChrM rates. The log10 of the rates were determined, which centralized the data, although the skewness still remained (FIG. 2B).

As shown in FIG. 2B, bottom panel, several healthy subjects with identical ChrM rates (three first bars) were identified. These rates were translated to the scatter plot (FIG. 2B, top panel), identifying different cancer samples with the same values. Although there is a possibility that these samples have identical rates, it is unlikely that two different subjects in different sequencing runs have the same rate. Another potential problem this might pose is that it may does not provide sufficient resolution to differentiate cancer from non-cancer samples.

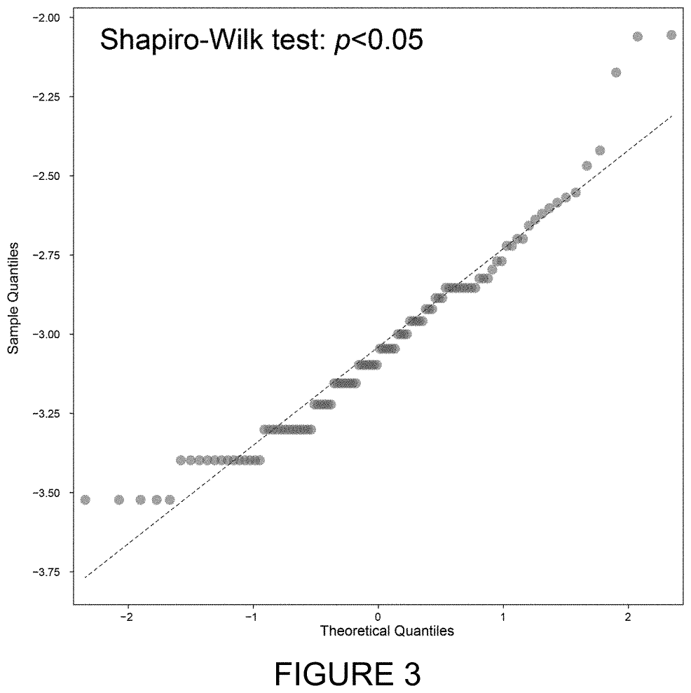

To make a determination of whether the log10 ChrM rate distribution was normal, a statistical analysis was provided. As shown in FIG. 3, a Q-Q plot provided an inference of whether the log10 ChrM rate distribution was normal. As shown in FIG. 3, the statistical data was close to the expected line, but some data points formed “steps” that deviated from the line. A combination with a normality test indicated that the distribution is significantly different from a normal distribution.

### Example 2

### Measuring ChrM Sequencing Depth

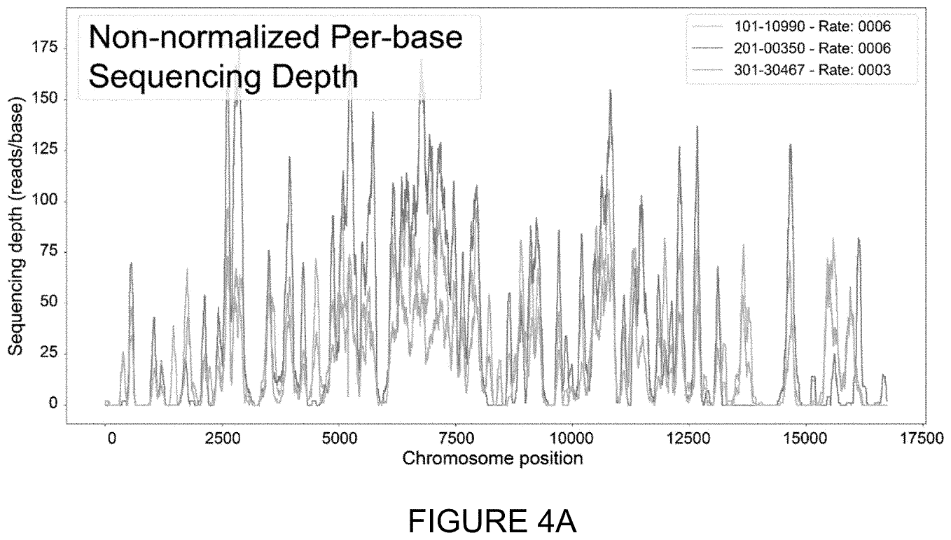

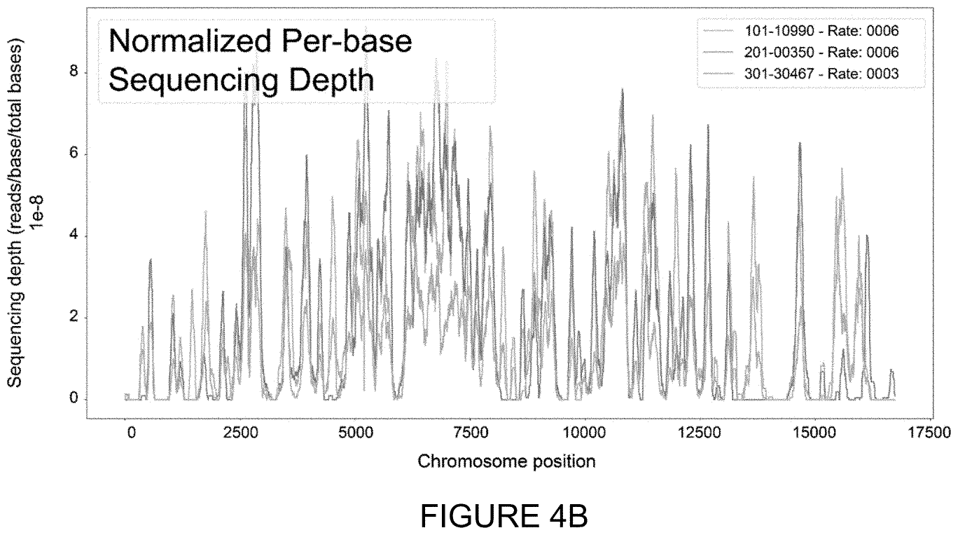

In order to separate samples with differences in the coverage for ChrM, the resolution was increased by counting the per-base sequencing depth instead of the number of mapped reads. As shown in FIGS. 4A and 4B, the per-base sequencing depth was plotted for three samples, as a function of chromosome position in a non-normalized (FIG. 4A) and normalized (FIG. 4B) method. The non-normalized sequencing depth included reads/base, and the normalized sequencing depth included reads/base/total bases. The plots in FIGS. 4A and 4B included three samples, two of which have identical ChrM rates of 0.006 (healthy sample 101 and cancer sample 201) and the third sample having a lower rate of 0.003 (cancer sample 301).

As shown in FIGS. 4A and 4B, the sequencing depth was constructed by counting the number of times each base along the ChrM was covered by a read. Thus, if two identical reads mapped to the same region, the bases covered would have a value of 2. This value was counted for all bases along the chromosome and the information plotted.

The per-base sequencing depth plots shown in FIGS. 4A and 4B indicated that two subjects that appeared identical in ChrM rates (samples 101 and 201) were not identical in their sequencing depth profiles. Indeed, sample 201 was more similar to sample 301 in sequencing depth, even though sample 301 had a lower ChrM rate. This supports that by measuring the sequencing depth, an improvement of resolution is achieved when comparing samples.

It was also evident from these sequencing depth plots that some regions in the chromosome were highly covered (for example close to position 2,500) and other regions had low to no coverage (for example before the peak at 15,000). Thus, by calculating the average sequencing depth, one can summarize the sequencing depth profile observed in FIG. 4A. Nonetheless, this average can be affected by the high- and low-coverage regions described above (extreme values).

In FIG. 4A, each position on the x-axis represents a base and the y-axis represents the sequencing depth of each base, or the number of times a base is covered by a read. Sample 201 consistently had the highest number of reads per base in almost every position. However, this was an artifact of the number of total reads mapping to ChrM, where sample 201 had the largest number of reads mapping to ChrM. By normalizing the sequencing depth by the total number of bases in the dataset (or by the number of total reads) as shown in FIG. 4B, the overall trend remained the same, but sample 101 had the highest sequencing depth in some peaks. In either case, sample 301 had the lowest sequencing depth over most positions, effectively maintaining the same trend that was observed in initial rate estimation (ChrM reads/total reads).

### Example 3

### Measuring Normalized Truncated Average Sequencing Depth (NTAD) to Identify Cancer Samples

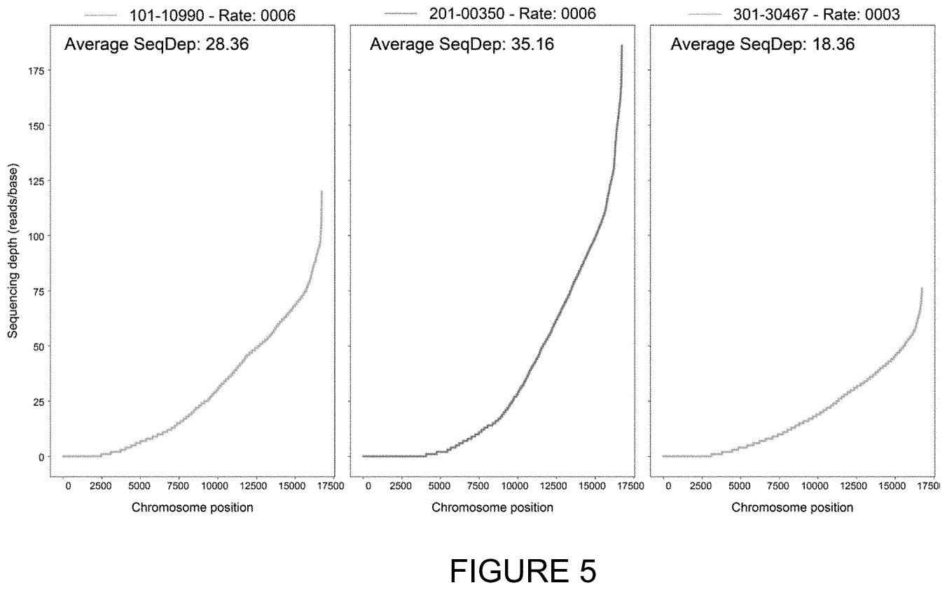

In order to measure whether there was an increase from bases with no coverage to highly covered bases, the per-base sequencing depth was sorted. As shown in FIG. 5, sample 201 (middle plot) had more extreme values that drove the average to higher values. Sample 101 had an average sequencing depth of 28.36; sample 201 had an average sequencing depth of 35.16; and sample 301 had an average sequencing depth of 18.36. Therefore, in order to reduce the effect of these extreme values, the ends of these distributions were removed and then the average sequencing depth was calculated.

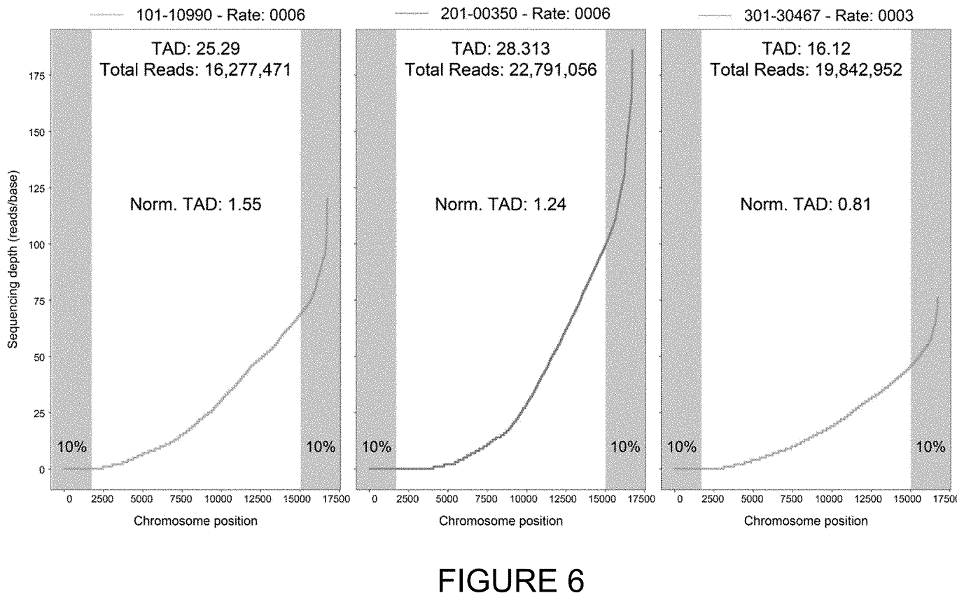

The data was organized by removing the top 10% and bottom 10% of distributions. As shown in FIG. 6, even with an increasing curve, the differences between the lowest and highest points were smaller. The truncated average sequencing depth (TAD) for sample 101 was 25.29; for sample 201 was 28.313; and for sample 301 was 16.12. The differences between these values decreased between TADs when no truncation was applied (6.9 vs 3.02). These TAD values were normalized (NTAD). For example, given that the TAD also depends on the size of the library, it was scaled by the number of total reads. Further, given that these numbers are usually small, they were scaled by a million reads. Thus, the NTAD were: 1.55 (sample 101); 1.24 (sample 201); and 0.81 (sample 301). These results were interpreted as the times the genome is covered per million reads.

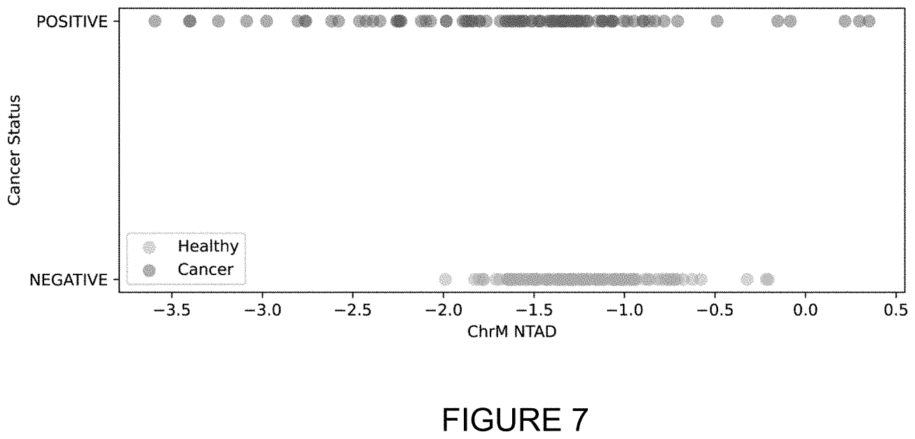

After calculating the NTADs in a training dataset, the healthy subjects (cancer status: NEGATIVE) were tighter and did not overlap at the lower end with the cancer subjects (cancer status: POSITIVE), as shown in FIG. 7. A more continuous distribution of values which help with the normality of the dataset was also observed.

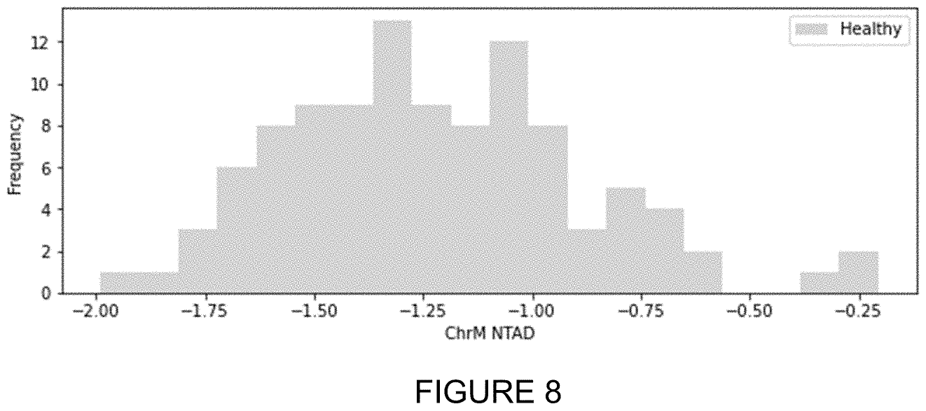

The distribution of NTADs for healthy subjects resembled a normal distribution, but with the presence of outliers, as shown in FIG. 8. Interestingly, all of these outliers had high fragment insert size standard deviations (>=400).

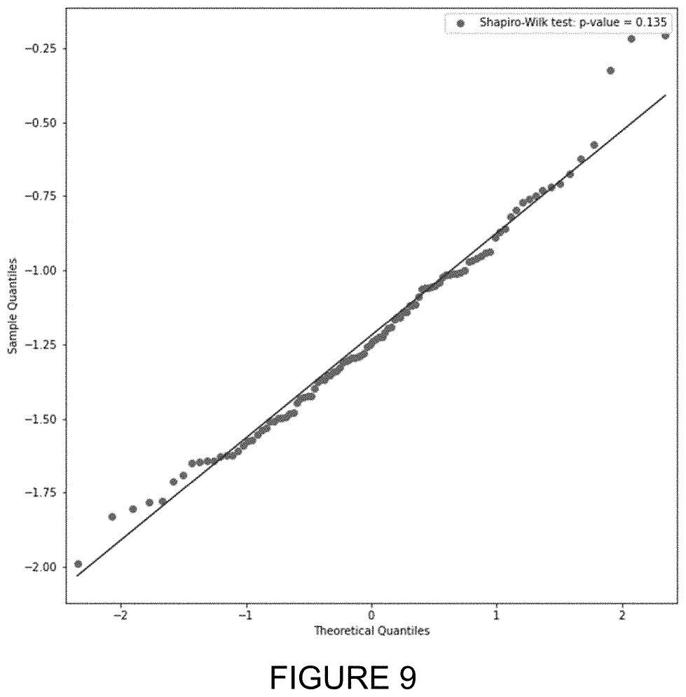

A Shapiro-Wilk test was performed, and a Q-Q plot was generated to ensure this distribution of NTAD values behaved normally, as shown in FIG. 9. Both approaches indicated normality, which means that thresholds can be established based on means and standard deviations or quantiles of a fitted distribution.

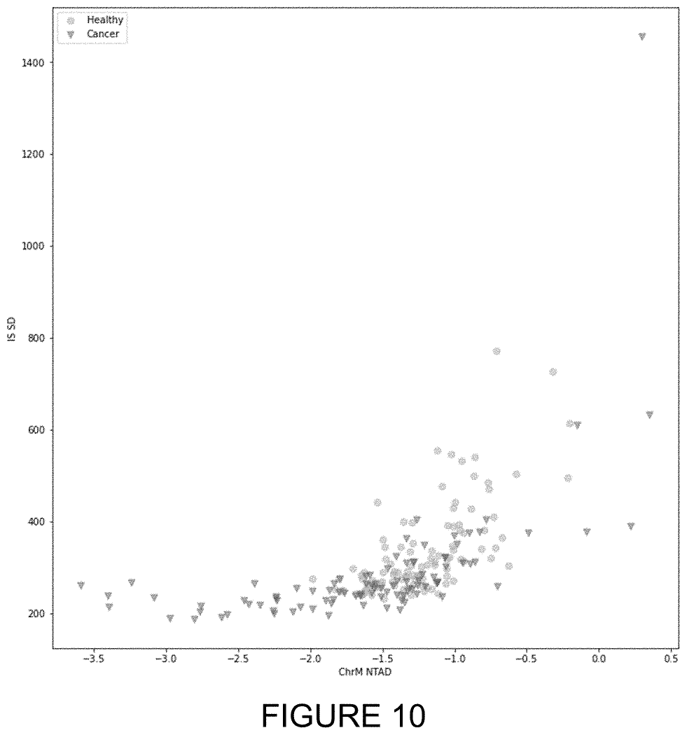

Given that outliers shared a high fragment insert size standard deviation (IS SD), the relation between NTAD values and IS SD was assessed as shown in FIG. 10. The scatter plot indicates that higher IS SD values are associated with higher NTAD values; this allowed the incorporation of an additional filter to remove samples with high IS SD values.

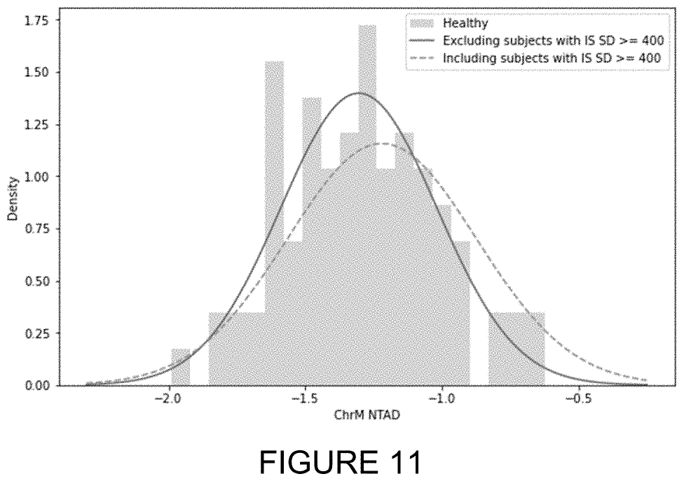

After the removal of samples with high fragment insert size standard deviation (IS SD), the histogram of healthy subjects in FIG. 11 indicated a tighter distribution. Indeed, the fitted normal distribution that included all subjects, represented by the dashed line in FIG. 11, had a larger mean and larger standard deviation compared to the fitted normal distribution of subjects with IS SD lower than 400 (solid line in FIG. 11).

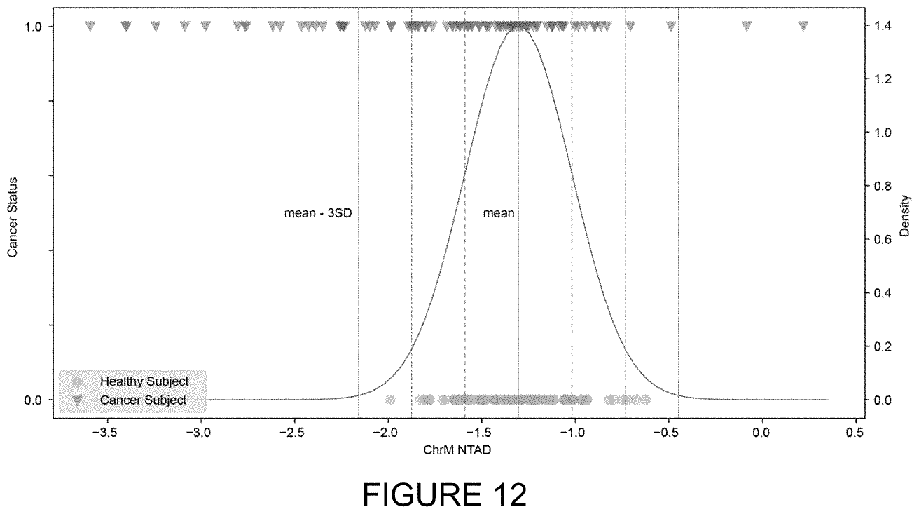

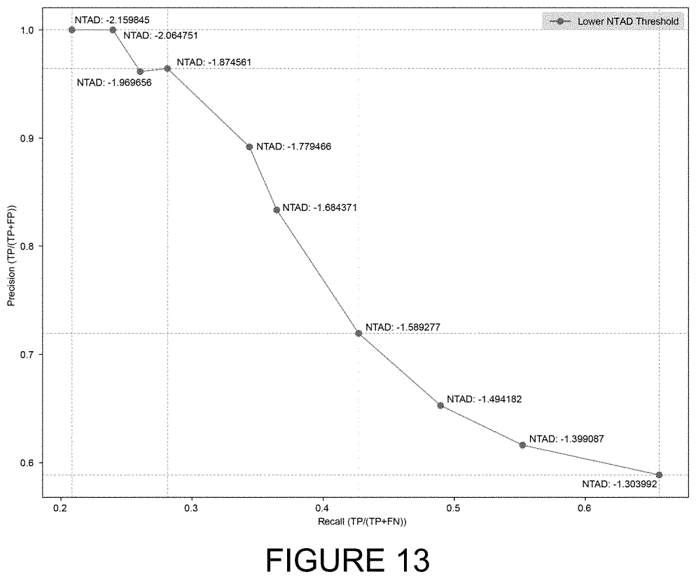

Accordingly, the mean of the healthy subjects (solid black line) and one, two, and three standard deviations above and below the mean were estimated from the normal fitted distribution of subjects with low fragment insert size standard deviation, as shown in FIG. 12. These thresholds were tested on the whole training dataset calculating precision-recall curves (FIG. 13). The precision and recall curve showed that a lower threshold established at a value of mean minus three standard deviations resulted in a precision of 1 (no false positives), while allowing the identification of cancer positive samples.

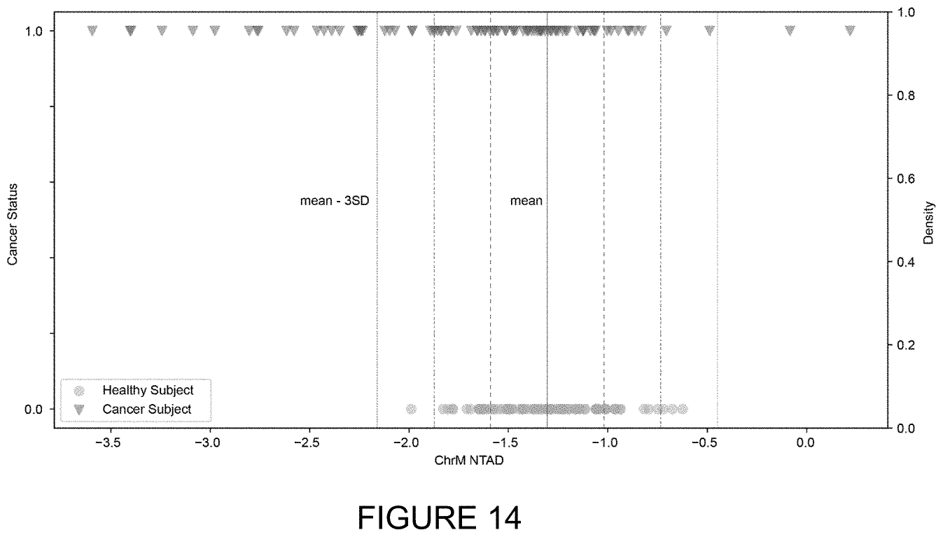

The lowest NTAD threshold (mean −3 SD) was used in the training dataset and allowed the identification of cancer positive samples, as shown in FIG. 14. Interestingly, this method resulted in the identification of cancer positive samples that were previously unable to be identified using complementary methods such as copy number variation (CNV) or fragmentomics analyses. However, as FIG. 14 also shows, the threshold established at a value of mean −3 SD may be too close to the lowest NTAD value observed for healthy subjects, which can potentially result in false positives if a slightly lower value in a normal, healthy subject is observed.

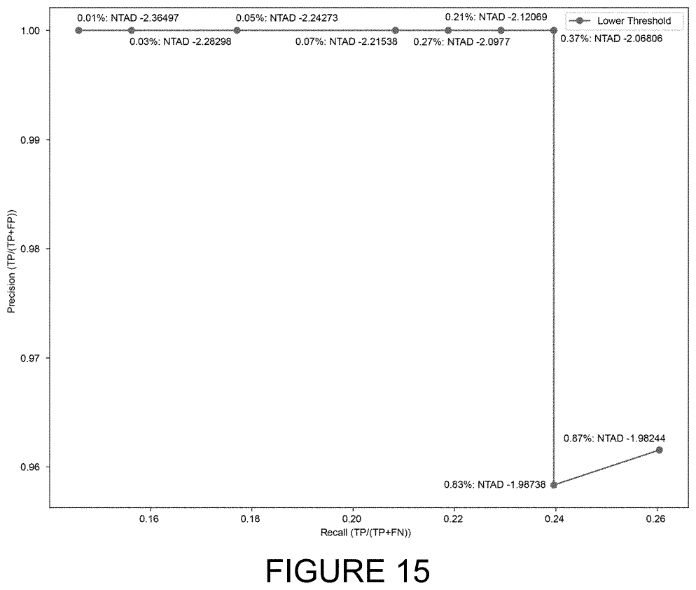

Therefore, lower thresholds established based on quantiles of the fitted normal distributions were also tested. FIG. 15 shows a plot of the precision/recall values observed when the threshold was determined based on quantiles from the normal distribution that ranged from 0.0001 to 0.01 (corresponding to NTAD values between −2.36497 and −1.98244). In some embodiments, the lowest threshold tested at NTAD=−2.36497 (corresponding to a 0.0001 quantile) provides a similar behavior as thresholds established based on the mean and standard deviations of the normal distribution, resulting in no false positives. Setting this lower threshold of greater was also useful for the identification of a cancer positive samples. Aiming to maximize the maximum theoretical precision, a threshold based on the 0.0001 quantile was established as the lower threshold.

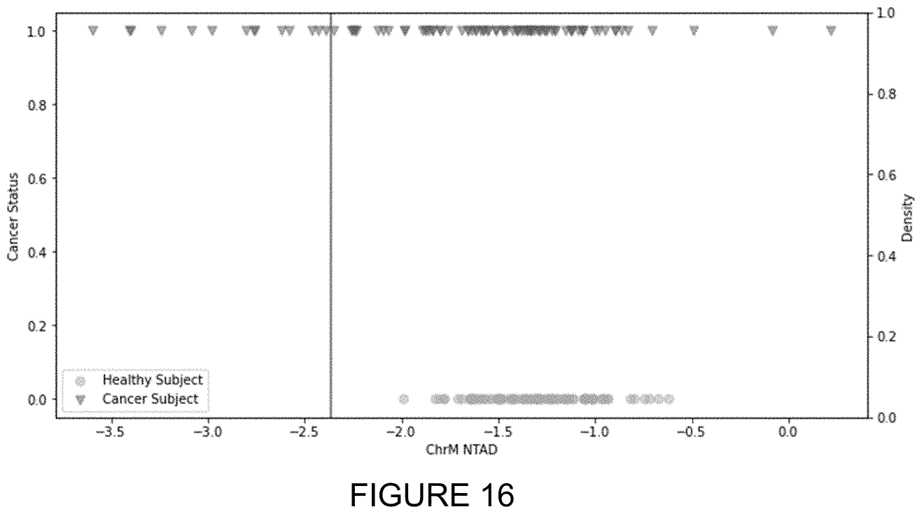

Consequently, as FIG. 16 shows, the use of a stricter threshold determined based on the lowest quantile tested also allowed the identification of cancer positive samples, even some that were not identified using complementary methods. At the same time, the stricter threshold resulted in additional separation from the lowest NTAD value from the healthy subjects group. This will permit the possibility of a wider spread in the distribution of NTAD values from future subjects while decreasing the potential for false positives.

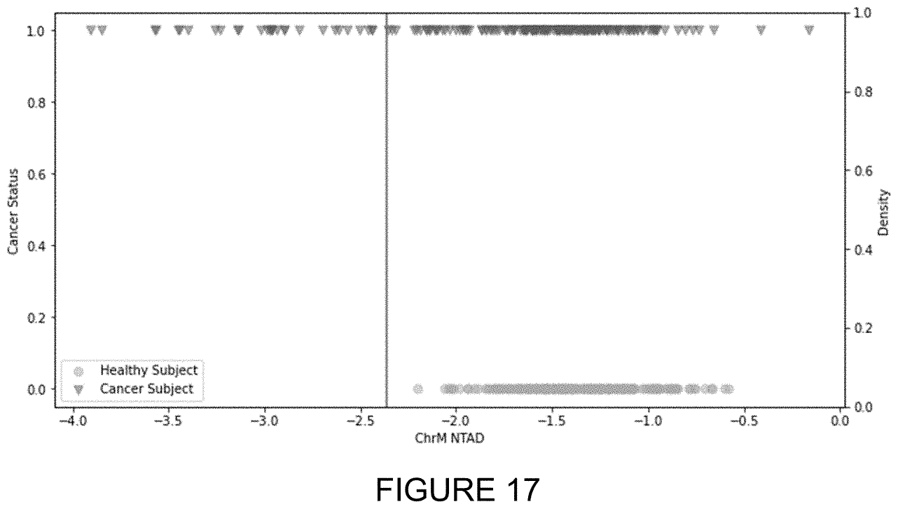

The distribution of NTAD values from healthy and cancer subject in the testing dataset was very similar to the one observed in the training dataset (FIG. 17). The threshold established above at NTAD=−2.36497 also resulted in a clear separation from healthy subjects (cancer status 0) and several cancer positive subjects (cancer status 1).

TABLE 1
Quantiles derived from the modeled normal distribution evaluated
as thresholds along with their corresponding NTAD values, the
number of true positives (TP), false positives (FP), true negatives
(TN), false negatives (FN), and new true positives (NTP) detected
in a training dataset composed of 200 subjects.

| Quantile | NTAD | TP | FP | TN | FN |
|---|---|---|---|---|---|
| 0.0001 | −2.36497 | 14 | 0 | 104 | 82 |
| 0.0002 | −2.313923 | 15 | 0 | 104 | 81 |
| 0.0003 | −2.282978 | 15 | 0 | 104 | 81 |
| 0.0004 | −2.260492 | 15 | 0 | 104 | 81 |
| 0.0005 | −2.242728 | 17 | 0 | 104 | 79 |
| 0.0006 | −2.227994 | 20 | 0 | 104 | 76 |
| 0.0007 | −2.215376 | 20 | 0 | 104 | 76 |
| 0.0008 | −2.204323 | 20 | 0 | 104 | 76 |
| 0.0009 | −2.194476 | 20 | 0 | 104 | 76 |
| 0.001 | −2.185587 | 20 | 0 | 104 | 76 |
| 0.0011 | −2.17748 | 20 | 0 | 104 | 76 |
| 0.0012 | −2.170022 | 20 | 0 | 104 | 76 |
| 0.0013 | −2.163113 | 20 | 0 | 104 | 76 |
| 0.0014 | −2.156674 | 20 | 0 | 104 | 76 |
| 0.0015 | −2.150642 | 20 | 0 | 104 | 76 |
| 0.0016 | −2.144966 | 20 | 0 | 104 | 76 |
| 0.0017 | −2.139604 | 20 | 0 | 104 | 76 |
| 0.0018 | −2.134523 | 20 | 0 | 104 | 76 |
| 0.0019 | −2.129692 | 20 | 0 | 104 | 76 |
| 0.002 | −2.125087 | 20 | 0 | 104 | 76 |
| 0.0021 | −2.120686 | 21 | 0 | 104 | 75 |
| 0.0022 | −2.116472 | 21 | 0 | 104 | 75 |
| 0.0023 | −2.112427 | 21 | 0 | 104 | 75 |
| 0.0024 | −2.108539 | 21 | 0 | 104 | 75 |
| 0.0025 | −2.104795 | 21 | 0 | 104 | 75 |
| 0.0026 | −2.101184 | 21 | 0 | 104 | 75 |
| 0.0027 | −2.097696 | 22 | 0 | 104 | 74 |
| 0.0028 | −2.094323 | 22 | 0 | 104 | 74 |
| 0.0029 | −2.091057 | 22 | 0 | 104 | 74 |
| 0.003 | −2.087891 | 22 | 0 | 104 | 74 |
| 0.0031 | −2.084819 | 22 | 0 | 104 | 74 |
| 0.0032 | −2.081835 | 22 | 0 | 104 | 74 |
| 0.0033 | −2.078933 | 22 | 0 | 104 | 74 |
| 0.0034 | −2.07611 | 22 | 0 | 104 | 74 |
| 0.0035 | −2.07336 | 22 | 0 | 104 | 74 |
| 0.0036 | −2.07068 | 23 | 0 | 104 | 73 |
| 0.0037 | −2.068065 | 23 | 0 | 104 | 73 |
| 0.0038 | −2.065514 | 23 | 0 | 104 | 73 |
| 0.0039 | −2.063022 | 23 | 0 | 104 | 73 |
| 0.004 | −2.060586 | 23 | 0 | 104 | 73 |
| 0.0041 | −2.058205 | 23 | 0 | 104 | 73 |
| 0.0042 | −2.055875 | 23 | 0 | 104 | 73 |
| 0.0043 | −2.053594 | 23 | 0 | 104 | 73 |
| 0.0044 | −2.05136 | 23 | 0 | 104 | 73 |
| 0.0045 | −2.04917 | 23 | 0 | 104 | 73 |
| 0.0046 | −2.047024 | 23 | 0 | 104 | 73 |
| 0.0047 | −2.044919 | 23 | 0 | 104 | 73 |
| 0.0048 | −2.042854 | 23 | 0 | 104 | 73 |
| 0.0049 | −2.040827 | 23 | 0 | 104 | 73 |
| 0.005 | −2.038836 | 23 | 0 | 104 | 73 |
| 0.0051 | −2.036881 | 23 | 0 | 104 | 73 |
| 0.0052 | −2.034959 | 23 | 0 | 104 | 73 |
| 0.0053 | −2.03307 | 23 | 0 | 104 | 73 |
| 0.0054 | −2.031212 | 23 | 0 | 104 | 73 |
| 0.0055 | −2.029385 | 23 | 0 | 104 | 73 |
| 0.0056 | −2.027586 | 23 | 0 | 104 | 73 |
| 0.0057 | −2.025817 | 23 | 0 | 104 | 73 |
| 0.0058 | −2.024074 | 23 | 0 | 104 | 73 |
| 0.0059 | −2.022358 | 23 | 0 | 104 | 73 |

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose, including the disclosures specifically referenced herein. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this invention has been disclosed in the context of certain embodiments and examples, those skilled in the art will understand that the present invention extends beyond the specifically disclosed embodiments to other alternative embodiments and/or uses of the invention and obvious modifications and equivalents thereof In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes or embodiments of the disclosed invention. Thus, it is intended that the scope of the present invention herein disclosed should not be limited by the particular disclosed embodiments described above.

It should be understood, however, that this detailed description, while indicating preferred embodiments of the invention, is given by way of illustration only, since various changes and modifications within the spirit and scope of the invention will become apparent to those skilled in the art.

The terminology used in the description presented herein is not intended to be interpreted in any limited or restrictive manner. Rather, the terminology is simply being utilized in conjunction with a detailed description of embodiments of the systems, methods, and related components. Furthermore, embodiments may comprise several novel features, no single one of which is solely responsible for its desirable attributes or is believed to be essential to practicing the inventions herein described.

What is claimed is:

1. A method of detecting cancer in a subject, comprising:
collecting a liquid biopsy sample from the subject;
determining ChrM sequencing depth;
truncating the ChrM sequencing depth;
calculating a relative quantity of ChrM DNA by comparison to total cfDNA; and
applying a threshold,
wherein applying the threshold isolates a subject with cancer from a cancer free subject.

2. The method of claim 1, wherein the relative quantity of ChrM DNA is a normalized truncated average sequencing depth (NTAD).

3. The method of claim 1, wherein the ChrM sequencing depth is an average sequencing depth.

4. The method of claim 1, wherein the relative quantity of ChrM DNA is a ChrM rate.

5. The method of claim 2, further comprising measuring log10 of NTAD.

6. The method of claim 5, wherein the NTAD is scaled by a factor prior to log10 transformation.

7. The method of claim 6, wherein the scale factor is 10, 100, 1,000, 10,000, 100,000, or 1,000,000.

8. The method of claim 1, wherein the ChrM sequencing depth is normalized by measuring ChrM reads per base per total reads.

9. The method of claim 3, wherein the ChrM average sequencing depth is normalized by measuring ChrM sequencing depth per base per total reads.

10. The method of claim 1, wherein truncating the ChrM sequencing depth comprises removing outliers from a distribution of ChrM per-base sequencing depth.

11. The method of claim 10, wherein the outliers comprise a top 10% and a bottom 10% of measured ChrM per-base sequencing depth.

12. The method of claim 2, wherein the threshold is 1, 2, or 3 standard deviations from the mean value of NTAD of healthy subjects.

13. The method of claim 2, wherein the threshold is more than 3 standard deviations from the mean value of NTAD of healthy subjects.

14. The method of claim 1, wherein the threshold is based on a modeled cumulative distribution function (CDF) quantile.

15. The method of claim 14, wherein the modeled CDF quantile is 0.99, 0.995, or

0. 999.

16. The method of claim 1, further comprising applying a statistical analysis to determine whether the relative quantity of ChrM DNA is distributed normally.

17. The method of claim 16, wherein the statistical analysis is a Q-Q test or a Shapiro-Wilk test.

18. The method of claim 1, further comprising determining precision/recall to determine a performance of the method and/or a relation between true positives, false positives, true negatives, and false negatives.

19. The method of claim 1, wherein the sample comprises circulating cell free DNA (cfDNA) or fragments thereof.

20. The method of claim 1, wherein the cancer sample is leukemia, lymphoma, testicular tumor, spinal meningioma, multilobular osteochondrosarcoma, soft tissue sarcoma, squamous cell carcinoma, mammary cancer, mast cell tumors, bladder cancer, osteosarcoma, or hemangiosarcoma.

21. The method of claim 1, wherein the subject is a mammal.

22. The method of claim 21, wherein the subject is canine, feline, equine, or human.
